(19) United States
(12) Patent Application Publication
(10) Pub. No.: US 2022/0226302 A1
(43) Pub. Date: Jul. 21, 2022

(54) ENHANCEMENT OF CAMP SIGNALING AS A COMBINATION DRUG STRATEGY FOR THE TREATMENT OF DEPRESSION AND RELATED CONDITIONS

(71) Applicant: Altos Neuroscience, Inc., Los Altos, CA (US)

(72) Inventors: Amit Etkin, Los Altos, CA (US); Dan Segal, Sydney (AU); Maria Ferreira da Silva, Los Altos, CA (US); Lauren Blake, Los Altos, CA (US)

(21) Appl. No.: 17/648,539

(22) Filed: Jan. 20, 2022

Related U.S. Application Data
(60) Provisional application No. 63/199,728, filed on Jan. 20, 2021.

(51) Int. Cl. A61K 31/454; A61K 31/501; A61K 31/439; A61K 31/216; A61K 31/381; A61K 31/5377

(52) U.S. Cl. CPC A61K 31/454; A61K 31/501; A61K 31/5377; A61K 31/216; A61K 31/381; A61K 31/439

(57) ABSTRACT

The present invention relates to the use of a combination of a phosphodiesterase 4 (PDE4) inhibitor and one or more of 5-HT₄ agonist, an H₃ antagonist or inverse agonist, a nicotinic α₇ receptor agonist, a β₃ adrenergic agonist or a TAAR1 agonist for the treatment of psychiatric or neurological disorders in which depressive, anhedonia, motivation-related or cognition-related dysfunction exists (such as major depressive disorder, bipolar I disorder, post-traumatic stress disorder, addiction, anhedonia or motivation-related aspects of schizophrenia (e.g. negative and cognitive symptoms), as well as Parkinson's disease (e.g. non-motor features such as depression, apathy and cognitive impairment)).

This application claims priority to U.S. Provisional Application No. 63/199,728 filed Jan. 20, 2021, the entire contents of which are hereby incorporated by reference herein.

### FIELD OF THE INVENTION

The present invention relates to the use of a combination of a phosphodiesterase 4 inhibitor and one or more of a 5-HT₄ agonist, an H₃ antagonist or inverse agonist, a nicotinic α₇ receptor agonist, a β₃ adrenergic agonist or a TAAR1 agonist for the treatment of psychiatric and neurological disorders in which depressive, anhedonia, motivation-related or cognition-related dysfunction exists (such as major depressive disorder, bipolar I disorder, post-traumatic stress disorder, addiction, anhedonia or motivation-related aspects of schizophrenia (e.g. negative and cognitive symptoms), as well as Parkinson's disease (e.g. non-motor features such depression, apathy and/or cognitive impairment).

### BACKGROUND

There are many psychiatric and neurological disorders in which depressive symptoms, lack of experience of pleasure (anhedonia) or motivation-related, and/or cognition-related impairments exist. These are often difficult-to-treat features of these diseases and are predictive of a chronic and disabling course of illness. A need exists for more effective treatment in depression and diseases or disorders with a depressive, anhedonia, motivational and/or cognitive impairment component, given that even with the most comprehensive treatment regimen, only 43% of depressed patients achieve sustained remission over a one-year period (1) and this is the disorder in which the largest number of treatment options exist. The psychiatric and neurological conditions featuring depressive, anhedonia, motivational and/or cognitive impairment symptoms include major depressive disorder, bipolar depression (such as bipolar I disorder), post-traumatic stress disorder (PTSD), addiction, schizophrenia (e.g., negative symptoms) and Parkinson's disease (e.g. non-motor features such as depression, apathy and cognitive impairment).

Importantly, these different depression-related symptoms or areas of dysfunction co-occur and may be functionally related. For example, a patient with major depression may report depressed mood, anhedonia, lack of motivation and cognitive difficulties. Likewise, the same symptoms may be reported by patients diagnosed with other conditions in which similar impairments may co-occur, such as bipolar depression, post-traumatic stress disorder or addiction. Though schizophrenia is often thought of with respect to prominent hallucinations and delusions, the depression-like negative symptoms and related cognitive symptoms are often the greater source of long-term disability and functional impairment. Similarly, though Parkinson's disease involves prominent motor dysfunction, it also frequently has highly disabling non-motor features such as depression, apathy and cognitive impairment. Hence, a treatment approach that encompasses these multiple and related functional systems would be both of importance to any one of these clinical conditions, and equally may be applicable across them.

Preclinical and clinical studies have suggested a role for cyclic adenosine monophosphate (cAMP) signaling as important for depression or aspects of its symptoms, which includes anhedonia, motivational dysfunction and cognitive impairment. For example, PET imaging of the binding and activity of phosphodiesterase 4 (PDE4), one of the important enzymes that breaks down cAMP, showed a reduction in PDE4 activity in patients with major depression (2). This has been interpreted as a decrease in cAMP signaling at baseline, which increases after antidepressant treatment given the tight relationship between cAMP levels, downstream effects on protein kinase A activity, and consequently the phosphorylation and enzymatic activity level of PDE4 (as a self-regulatory feedback loop). Consistent with this view, injection of an inhibitor of protein kinase A into the brain resulted in a decrease in PET-measured PDE4 levels, while injection of a protein kinase A activator resulted in an increase in PET-measured PDE4 levels (3). Post-mortem studies have found alterations in other elements of cAMP-related signaling, such as adenylyl cyclase levels (4-6). A role for dysfunction in cAMP signaling has also been proposed observed in other disorders in which depressive or cognition-related dysfunction exists (such as, bipolar disorder and schizophrenia) (27-28).

Given these findings, one of the primary ways to increase cAMP signaling pharmacologically has been to inhibit its breakdown, with the primary focus on the role of PDE4. This protein is present across much of the nervous system (as well as in various peripheral tissues), making its manipulation potentially of therapeutic impact. Mice lacking subtypes of the PDE4 protein display both antidepressant-like and pro-cognitive phenotypes (7). Similarly, mice treated with rolipram, a canonical inhibitor of PDE4, show similar antidepressant and pro-cognitive phenotypes (7). The utility of PDE4 inhibitors in humans for the treatment of depression is far less clear. To date, there has not been a single placebo-controlled study of any PDE4 inhibitor in patients with depression. Likewise, there have not been any placebo-controlled study of a PDE4 inhibitor in other psychiatric disorders in which depressive, anhedonia, motivation-related or cognition-related dysfunction exists (such as major depressive disorder, bipolar I disorder, post-traumatic stress disorder, addiction and anhedonia or motivation-related aspects of schizophrenia (e.g. negative and cognitive symptoms)). There have been a number of small clinical trials in depression that have compared the PDE4 rolipram to tricyclic antidepressant (TCA) medications (8-10). In none of these studies, however, did rolipram produce a greater improvement in depression than the TCA medications, and in several instances lead to significantly worse outcomes (9,10). Moreover, even when outcomes were not statistically distinguishable with rolipram from a TCA, this was in studies far too underpowered to demonstrate non-inferiority with any statistical confidence. Roflumilast, another PDE4 inhibitor that had been developed for chronic obstructive pulmonary disease, which can also reach the brain, has been found to have pro-cognitive effects broadly consistent with work on PDE4 inhibitors in animals (11-13), but there has been no evidence of an antidepressant effect.

Despite the promise for the therapeutic potential of PDE4 inhibitors for depression and related conditions, they have been held back by a limited therapeutic index due to dose-limiting side effects such as nausea and vomiting. Indeed, this limitation is so severe as to affect all clinically tested PDE4 inhibitors and occurs at such a relatively low dose (relative to percent target occupancy) so that the ultimate range between being ineffective and intolerable is too small to allow the drug to be meaningfully used for the treatment of neuropsychiatric conditions. Put differently, part of the power and promise of boosting cAMP signaling is that this second messenger is used across the entire brain, and thus has potential to improve many aspects of neuropsychiatric illnesses. But this virtue is also its main downside, as cAMP in areas such as the area postrema in the brainstem also mediates the emetic effects of PDE4 inhibitors (14). Thus far it has not been possible to achieve a potent increase in cAMP signaling in parts of the brain important for depression and related conditions using a PDE4 inhibitor (e.g. frontal cortex, hippocampus and striatum) while avoiding doing so in emesis-causing areas such as the area postrema. This has resulted in multiple brain directed PDE4 inhibitor development initiatives being terminated by pharmaceutical companies.

The principal suggestion for how the promise of PDE4 inhibition can be realized, that is by minimizing its substantial side effects, is that new drugs can be designed that inhibit particular isoforms of the protein which are more important for its therapeutic potential relative to its side effects (15, 16). Doing so, however, requires that different isoforms both have functions that can be dissociated in that way, or that small molecule inhibitor drugs can selectively target PDE4 in particular brain locations. To date, no such drugs have been identified and advanced to testing in humans. In this invention, we view this challenge differently, and detail a novel solution.

### SUMMARY OF THE INVENTION

One embodiment of the present invention is a pharmaceutical composition (e.g., an oral composition such as an oral tablet or oral solution) comprising a PDE4 inhibitor (such as roflumilast, its N-oxide, or a pharmaceutically acceptable salt thereof) and at least one of a 5-HT₄ agonist, an H₃ antagonist or inverse agonist, a nicotinic α₇ receptor agonist, a β₃ adrenergic agonist or a TAAR1 agonist.

The composition may comprise (a) a PDE4 inhibitor and (b) a 5-HT₄ agonist. In one embodiment, the composition comprises (a) roflumilast, its N-oxide, or a pharmaceutically acceptable salt thereof and (b) prucalopride or a pharmaceutically acceptable salt thereof (such as prucalopride succinate). For instance, the composition may comprise (a) from about 100 to about 500 mcg of roflumilast or the equivalent amount of a pharmaceutically acceptable salt of roflumilast and (b) from about 0.25 to about 4 mg of prucalopride or the equivalent amount of a pharmaceutically acceptable salt of prucalopride (such as prucalopride succinate). In another embodiment, the composition comprises (a) roflumilast, its N-oxide, or a pharmaceutically acceptable salt thereof and (b) capeserod or a pharmaceutically acceptable salt thereof (such as capeserod hydrochloride). For instance, the composition may comprise (a) from about 100 to about 500 mcg of roflumilast or the equivalent amount of a pharmaceutically acceptable salt of roflumilast and (b) from about 1 μg to about 10 mg of capeserod or the equivalent amount of a pharmaceutically acceptable salt of capeserod (such as capeserod hydrochloride).

In another embodiment, the composition comprises (a) a PDE4 inhibitor and (b) an H₃ antagonist or inverse agonist. In one embodiment, the composition comprises (a) roflumilast, its N-oxide, or a pharmaceutically acceptable salt thereof and (b) pitolisant or a pharmaceutically acceptable salt thereof (such as pitolisant hydrochloride). For instance, the composition may comprise (a) from about 100 to about 500 mcg of roflumilast or the equivalent amount of a pharmaceutically acceptable salt of roflumilast and (b) an amount of pitolisant or a pharmaceutically acceptable salt thereof (such as pitolisant hydrochloride) equivalent to about 2 to about 40 mg of pitolisant hydrochloride. In another embodiment, the composition comprises (a) roflumilast, its N-oxide, or a pharmaceutically acceptable salt thereof and (b) irdabisant or a pharmaceutically acceptable salt thereof (such as irdabisant hydrochloride). For instance, the composition may comprise (a) from about 100 to about 500 mcg of roflumilast or the equivalent amount of a pharmaceutically acceptable salt of roflumilast and (b) an amount of irdabisant or a pharmaceutically acceptable salt thereof (such as irdabisant hydrochloride) equivalent to about 1 to about 500 μg of irdabisant hydrochloride.

In yet another embodiment, the composition comprises (a) a PDE4 inhibitor and (b) a nicotinic α₇ receptor agonist. In one embodiment, the composition comprises (a) roflumilast, its N-oxide, or a pharmaceutically acceptable salt thereof and (b) varenicline or a pharmaceutically acceptable salt thereof. For instance, the composition may comprise (a) from about 100 to about 500 mcg of roflumilast or the equivalent amount of a pharmaceutically acceptable salt of roflumilast and (b) from about 0.25 to about 3 mg of varenicline or the equivalent amount of a pharmaceutically acceptable salt thereof (such as varenicline tartrate).

In yet another embodiment, the composition comprises (a) a PDE4 inhibitor and (b) a β₃ adrenergic agonist. In one embodiment, the composition comprises (a) roflumilast, its N-oxide, or a pharmaceutically acceptable salt thereof and (b) amibegron or a pharmaceutically acceptable salt thereof. For instance, the composition may comprise (a) from about 100 to about 500 mcg of roflumilast or the equivalent amount of a pharmaceutically acceptable salt of roflumilast and (b) from about 100 to about 1400 mg of amibegron or the equivalent amount of a pharmaceutically acceptable salt thereof (e.g., amibegron hydrochloride).

In yet another embodiment, the composition comprises (a) a PDE4 inhibitor and (b) a TAAR1 agonist. In one embodiment, the composition comprises (a) roflumilast, its N-oxide, or a pharmaceutically acceptable salt thereof and (b) ulotaront (SEP-363856) or a pharmaceutically acceptable salt thereof. For instance, the composition may comprise (a) from about 100 to about 500 mcg of roflumilast or the equivalent amount of a pharmaceutically acceptable salt of roflumilast and (b) from about 5 to about 200 mg of ulotaront (SEP-363856) or the equivalent amount of a pharmaceutically acceptable salt thereof. In another embodiment, the composition comprises (a) roflumilast, its N-oxide, or a pharmaceutically acceptable salt thereof and (b) ralmitaront (RO6889450) or a pharmaceutically acceptable salt thereof. For instance, the composition comprises (a) from about 100 to about 500 mcg of roflumilast or the equivalent amount of a pharmaceutically acceptable salt of roflumilast and (b) from about 5 to about 300 mg of ralmitaront (RO6889450) or the equivalent amount of a pharmaceutically acceptable salt thereof.

In any of the embodiments described herein, the pharmaceutical composition may include a sub-emetic amount of component (a) (the PDE4 inhibitor).

In any of the embodiments described herein, the pharmaceutical composition may include a sub-emetic amount of component (a) and an effective amount of components (a) and (b) together to treat the intended disorder, such as (a) depression (such as major depressive disorder or bipolar I disorder), (b) a psychiatric or neurological disorder in which anhedonia, motivation-related or cognition-related dysfunction exists, or (c) one or more symptoms associated with depression, anhedonia, or motivation-related or cognition-related impairments.

In any of the embodiments described herein, the pharmaceutical composition may include an effective amount of the recited components (such as components (a) and (b)) to increase cAMP signaling.

Another embodiment is a method of treating (a) depression (such as major depressive disorder or bipolar I disorder), (b) a psychiatric or neurological disorder in which anhedonia, motivation-related or cognition-related dysfunction exists, or (c) one or more symptoms associated with depression, anhedonia, or motivation-related or cognition-related impairments in a subject in need thereof comprising administering to the subject an effective amount of a pharmaceutical composition of the present invention. In one embodiment, an effective amount of the pharmaceutical composition is administered to increase cAMP signaling. The psychiatric or neurological disorder can be post-traumatic stress disorder (PTSD), schizophrenia, addiction, or Parkinson's disease.

Yet another embodiment is a method of treating (a) depression (such as major depressive disorder or bipolar I disorder), (b) a psychiatric or neurological disorder in which anhedonia, motivation-related or cognition-related dysfunction exists, or (c) one or more symptoms associated with depression, anhedonia, or motivation-related or cognition-related impairments in a subject in need thereof comprising administering to the subject an effective amount of a PDE4 inhibitor (such as roflumilast, its N-oxide, or a pharmaceutically acceptable salt thereof) and at least one of a 5-HT₄ agonist, an H₃ antagonist or inverse agonist, a nicotinic α₇ receptor agonist, a β₃ adrenergic agonist or a TAAR1 agonist. The psychiatric or neurological disorder can be post-traumatic stress disorder (PTSD), schizophrenia, addiction, or Parkinson's disease.

In one embodiment, the method comprises administering an effective amount of (a) a PDE4 inhibitor and (b) a 5-HT₄ agonist. In one preferred embodiment, the method comprises administering an effective amount of (a) roflumilast, its N-oxide, or a pharmaceutically acceptable salt thereof and (b) prucalopride or a pharmaceutically acceptable salt thereof (such as prucalopride succinate). For instance, the method may comprise administering (a) from about 100 to about 500 mcg per day of roflumilast or the equivalent amount of a pharmaceutically acceptable salt of roflumilast and (b) from about 0.25 to about 4 mg per day of prucalopride or the equivalent amount of a pharmaceutically acceptable salt of prucalopride (such as prucalopride succinate). In another preferred embodiment, the method comprises administering an effective amount of (a) roflumilast, its N-oxide, or a pharmaceutically acceptable salt thereof and (b) capeserod or a pharmaceutically acceptable salt thereof (such as capeserod hydrochloride). For instance, the method may comprise administering (a) from about 100 to about 500 mcg per day of roflumilast or the equivalent amount of a pharmaceutically acceptable salt of roflumilast and (b) from about 1 μg to 10 mg of per day of capeserod or the equivalent amount of a pharmaceutically acceptable salt of capeserod (such as capeserod hydrochloride).

In another embodiment, the method comprises administering an effective amount of (a) a PDE4 inhibitor and (b) an H₃ antagonist or inverse agonist. In one preferred embodiment, the composition comprises administering an effective amount of (a) roflumilast, its N-oxide, or a pharmaceutically acceptable salt thereof and (b) pitolisant or a pharmaceutically acceptable salt thereof (such as pitolisant hydrochloride). For instance, the method may comprise administering (a) from about 100 to about 500 mcg per day of roflumilast or the equivalent amount of a pharmaceutically acceptable salt of roflumilast and (b) an amount of pitolisant or a pharmaceutically acceptable salt thereof (such as pitolisant hydrochloride) equivalent to about 2 to about 40 mg of pitolisant hydrochloride per day. In another preferred embodiment, the composition comprises administering an effective amount of (a) roflumilast, its N-oxide, or a pharmaceutically acceptable salt thereof and (b) irdabisant or a pharmaceutically acceptable salt thereof (such as irdabisant hydrochloride). For instance, the method may comprise administering (a) from about 100 to about 500 mcg per day of roflumilast or the equivalent amount of a pharmaceutically acceptable salt of roflumilast and (b) an amount of irdabisant or a pharmaceutically acceptable salt thereof (such as irdabisant hydrochloride) equivalent to about lug to about 500 μg of irdabisant hydrochloride per day.

In yet another embodiment, the method comprises administering an effective amount of (a) a PDE4 inhibitor and (b) a nicotinic α₇ receptor agonist. In one preferred embodiment, the composition comprises administering an effective amount of (a) roflumilast, its N-oxide, or a pharmaceutically acceptable salt thereof and (b) varenicline or a pharmaceutically acceptable salt thereof (such as varenicline tartrate). For instance, the method may comprise administering (a) from about 100 to about 500 mcg per day of roflumilast or the equivalent amount of a pharmaceutically acceptable salt of roflumilast and (b) from about 0.25 to about 3 mg per day of varenicline or the equivalent amount of a pharmaceutically acceptable salt of varenicline (such as varenicline tartrate).

In yet another embodiment, the method comprises administering an effective amount of (a) a PDE4 inhibitor and (b) a β₃ adrenergic agonist. In one preferred embodiment, the composition comprises administering an effective amount of (a) roflumilast, its N-oxide, or a pharmaceutically acceptable salt thereof and (b) amibegron or a pharmaceutically acceptable salt thereof (e.g., amibegron hydrochloride). For instance, the method may comprise administering (a) from about 100 to about 500 mcg per day of roflumilast or the equivalent amount of a pharmaceutically acceptable salt of roflumilast and (b) from about 100 to about 1400 mg per day of amibegron or the equivalent amount of a pharmaceutically acceptable salt thereof (e.g., amibegron hydrochloride).

In yet another embodiment, the method comprises administering an effective amount of (a) a PDE4 inhibitor and (b) a TAAR-1 agonist. In one preferred embodiment, the composition comprises administering an effective amount of (a) roflumilast, its N-oxide, or a pharmaceutically acceptable salt thereof and (b) ulotaront (SEP-363856) or a pharmaceutically acceptable salt thereof. For instance, the method may comprise administering (a) from about 100 to about 500 mcg per day of roflumilast or the equivalent amount of a pharmaceutically acceptable salt of roflumilast and (b) from about 5 to about 200 mg per day of ulotaront (SEP-363856) or the equivalent amount of a pharmaceutically acceptable salt thereof. In another preferred embodiment, the composition comprises administering an effective amount of (a) roflumilast, its N-oxide, or a pharmaceutically acceptable salt thereof and (b) ralmitaront (RO6889450) or a pharmaceutically acceptable salt thereof. For instance, the method may comprise administering (a) from about 100 to about 500 mcg per day of roflumilast or the equivalent amount of a pharmaceutically acceptable salt of roflumilast and (b) from about 5 to about 300 mg per day of ralmitaront (RO6889450) or the equivalent amount of a pharmaceutically acceptable salt thereof.

In another embodiment, the methods described herein may include administering an effective amount of the recited components (such as components (a) and (b)) to increase cAMP signaling.

A preferred PDE4 inhibitor in any of the compositions or methods described herein is roflumilast, AVE8112 (4-(cyclopropylmethoxy)-N-(3,5-dichloro-1-oxidopyridin-4-yl)-5-methoxypyridine-2-carboxamide), MEM1414, MEM1917, apremilast, cilomilast, crisaborole, ibudilast, luteolin, mesembrenon, piclamilast, rolipram, chlorbipram, GSK-256066, E-6005, MK-0873, BPN14770, HT-0712, or a pharmaceutically acceptable salt thereof. Other suitable PDE4 inhibitors include those disclosed in International Publication Nos. WO 95/04545 and WO 2008/145840, which are hereby incorporated by reference.

A preferred 5-HT₄ agonist in any of the compositions or methods described herein is prucalopride, cisapride, BIMU-8, CJ-033466, ML-10302, mosapride, renzapride, RS-67506, RS67333, SL65.0155 (capeserod), tegaserod, zacopride, metoclopramide, supride, or a pharmaceutically acceptable salt thereof (such as prucalopride succinate).

A preferred H₃ antagonist or H₃ inverse agonist in any of the compositions or methods described herein is pitolisant, ABT-28, BF2.649, CEP-26401(irdabisant), GSK-189254, GSK-239512, MK-0249, PF-3654746, or a pharmaceutically acceptable salt thereof (such as pitolisant hydrochloride).

A preferred nicotinic α₇ receptor agonist for the compositions and methods described herein is varenicline, tilorone, A-582941, AR-R17779, TC-1698, bradanicline, encenicline, GTS-21, PHA-543613, PNU-292987, PHA-709829, SSR-180711, tropisetron, WAY-317538, anabasine, epiboxidine, PNU-120596, NS-1738, AVL-3288, A867744, ivermectine, BNC210 or a pharmaceutically acceptable salt thereof (such as varenicline tartrate).

A preferred TAAR1 agonist for the compositions and methods described herein is ulotaront (SEP-363856), ralmitaront (RO6889450), RO5166017, RO5256390, RO5203648, RO5263397, tyramine, amphetamine, methamphetamine, 3,4-methylenedioxy-methamphetamine (MDMA), or a pharmaceutically acceptable salt thereof.

### BRIEF DESCRIPTION OF THE DRAWINGS

FIG. 1 includes a schematic of (A) desired complementary exposure for the drug combinations, compared to (B) drug targets that do not overlap the disease module or (C) drug targets that overlap the disease module and each other. Complementary exposure means that each drug module is proximal to different parts of the disease module (and by extension, the drug modules do not overlap each other).

FIG. 2 is a table (Table 1) showing the distance and proximity of the specified drugs to certain diseases and cognition modules. The modules targeted by drugs within the combinations are significantly closer to the disease and cognition modules than randomly selected, similarly sized modules. For all drugs, except the (33 adrenergic agonist, a network consisting of direct targets plus additional genes whose expression is perturbed upon treatment of neurons or neural precursor cells with the drug was used. For the (33 adrenergic agonist, direct targets only were used due to lack of gene expression data. In this table, negative Z-scores represent statistically significant results, as assessed by permutation tests.

FIG. 3 is a table (Table 2) showing that modules targeted by the drugs within the combinations are complementary to each other, within disease and cognition modules. For all drug combinations, except PDE4 inhibitor+(33 adrenergic agonist, a network consisting of direct targets plus additional genes whose expression is perturbed upon treatment of neurons or neural precursor cells with the drug was used. For the PDE4 inhibitor+(33 adrenergic agonist combination, direct targets only were used due to lack of gene expression data. A positive separation value indicates the two drug modules are significantly distant from each other.

FIG. 4 is a table (Table 3) showing the distance and proximity of the specified drugs to certain diseases and cognition modules. Predicted gene expression modules targeted by the drug combinations are significantly closer to the disease modules than randomly selected, similarly sized modules, supporting their indication in treating the corresponding conditions. Prediction of gene expression networks for a PDE4 inhibitor+(33 adrenergic agonist combination was not done due to lack of data. In this table, negative Z-scores represent statistically significant results, as assessed by permutation tests.

FIG. 5 is a table (Table 4) which is a summary of the evidence for the drug combinations. All combinations are indicated for either all or the majority of the conditions as assessed by the two methodologies. For the PDE4 inhibitor+(33 adrenergic agonist combination, there is no available gene expression data. Therefore, no assessment of this indication was performed with the perturbation network methodology.

### DETAILED DESCRIPTION OF THE INVENTION

Without being bound by any particular theory, the inventors theorize that a solution to this challenge is through combining a sub-emetic amount of a PDE4 inhibitor with a drug that has an additive or synergistic action with respect to increasing cAMP signaling. In other words, the goal of the drug combination is to result in an additive or greater effect through directly increasing cAMP levels while simultaneously having the PDE4 inhibitor prevent the breakdown of the cAMP that is induced. It has not previously been demonstrated that PDE4 inhibitors are effective antidepressants in humans, or that combination with any other drug increases the antidepressant effect over a PDE4 alone. As such, there is a high degree of uncertainty regarding whether a drug when administered in combination with a PDE4 can effectively treat depression. There are several significant benefits of the combination of the present invention. First, the net increase in cells targeted by the two drugs would be larger than that possible with either drug alone and at lower doses, thereby resulting in fewer side effects, side effects of reduced severity, or both. Second, in particular, use of a sub-emetic amount of the PDE4 inhibitor would ensure greater tolerability of the combination relative to the higher dose of a PDE4 inhibitor that would be otherwise required to reach the same level of cAMP increase. Third, by combining a PDE4 inhibitor with a drug that influences neurons located in a subset of brain regions (or cell types) that are subject to the ubiquitous nature of PDE4, greater specificity can be achieved. This last advantage is critical as it allows for greater cAMP elevation in a subset of cells, regions or systems such that therapeutic effect can be maximized while side effect potential is minimized. The inventors surprisingly discovered at least three novel drug combinations that can enhance cAMP signaling for the treatment of depression or related conditions.

5-HT₄ agonist and PDE4 inhibitor combination: One receptor whose activation results in an increase in cAMP levels is the serotonin 5-HT₄ receptor. Activation of the 5-HT₄ receptor with an agonist (such as a partial agonist) has resulted in antidepressant-like effects in animals (17), suggesting effects on the brain, though no 5-HT₄ agonist has been developed for the treatment of neuropsychiatric conditions. Because of the prevalence of 5-HT₄ receptors in the alimentary and urinary tracts, however, agonists for this receptor have been approved for indications such as irritable bowel syndrome, and constipation. In experiments with human or pig intestinal preparations, it has also been found that a combination of a PDE4 inhibitor and a 5-HT₄ agonist can result in synergistic or additive activation of neuromuscular neurons (18, 19). Moreover, brain-penetrant oral 5-HT₄ agonists have been reported (20). Though 5-HT₄ agonists, such as prucalopride, are not used for the treatment of depression (prucalopride is indicated for the treatment of constipation), here we disclose that the surprising combination of a 5-HT₄ agonist and a sub-emetic amount of a PDE4 inhibitor can be used to treat symptoms related to depression, anhedonia, motivation-related or cognitive impairments. For example, use of 0.25-4 mg of the 5-HT₄ agonist prucalopride or a pharmaceutically acceptable salt thereof (such as prucalopride succinate), or 1 μg-10 mg of the 5HT₄ agonist capeserod (SL65.0155) or a pharmaceutically acceptable salt thereof (such as capeserod hydrochloride), concurrently with 100-500 mcg (such as 100-400 mcg, 100-300 mcg, 100-250 mcg or 100-200 mcg) of the PDE4 inhibitor roflumilast or an equivalent amount of a pharmaceutically acceptable salt of roflumilast is one such combination. This combination can both lead to greater improvement in depressive, anhedonia, motivational or cognitive symptoms and/or lead to lower side effects compared to use of a PDE4 inhibitor such as roflumilast alone.

H₃ antagonist or inverse agonist and PDE4 inhibitor combination: In addition to 5-HT₄, the histamine H₃ receptor is also coupled to cAMP, though in this case its activation inhibits cAMP production via coupling to Gi proteins. As such, a drug that is an H₃ antagonist or inverse agonist would be expected to result in increased cAMP levels. Various such H₃ antagonist or inverse agonist drugs have been tested for a number of cognitive disorders such as Alzheimer's disease, dementia, schizophrenia, multiple sclerosis and attention-deficit hyperactivity disorder, as well as states of altered alertness such as narcolepsy and obstructive sleep apnea (21-23). No such drugs, however, have been used to treat depressive symptoms. Moreover, combination drugs including an H₃ antagonist or inverse agonist have not been tested for the treatment of the cognitive and alertness disorders listed above. One embodiment of the invention is a combination of an H₃ antagonist or inverse agonist with a sub-emetic amount of a PDE4 inhibitor for treatment of symptoms related to depression, anhedonia, motivation-related or cognitive impairments. An example of an H₃ antagonist or inverse agonist is pitolisant, which is approved for the treatment of narcolepsy. Thus, for example, 2-40 mg of pitolisant hydrochloride or an equivalent amount of pitolisant or a different pharmaceutically acceptable salt thereof, or 1-500 μg of irdabisant (CEP-26401) or a pharmaceutically acceptable salt thereof (such as irdabisant hydrochloride), concurrently with 100-500 mcg of the PDE4 inhibitor roflumilast or an equivalent amount of a pharmaceutically acceptable salt of roflumilast is one such combination. This combination can both lead to greater improvement in depressive, anhedonia, motivational or cognitive symptoms and/or lead to lower side effects compared to use of a PDE4 inhibitor such as roflumilast alone.

Nicotinic α₇ receptor agonist and PDE4 inhibitor combination: Without being bound by a particular theory, the inventors hypothesized that a drug which agonizes the nicotinic α₇ receptor when combined with a PDE4 inhibitor can result in an additive or synergistic increase in cAMP levels. It has been recently reported that activation of the nicotinic α₇ receptor in the hippocampus results in an increase in cAMP levels by virtue of secondary effects on adenylyl cyclase, which produces cAMP (24). No nicotinic α₇ receptor agonist, however, has been used for the treatment of depression, one example of which is varenicline, which is approved for smoking cessation. Of note, however, varenicline has been approved in humans for smoking cessation, and initially carried a black box for increased risk for development of depression, a seemingly opposite outcome to our goal. One embodiment of the invention is a combination of a nicotinic α₇ receptor agonist with a sub-emetic amount of a PDE4 inhibitor for treatment of symptoms related to depression, anhedonia, motivation-related or cognitive impairments. Thus, for example, 0.25-3 mg of varenicline or an equivalent amount of a pharmaceutically acceptable salt of varenicline (such as varenicline tartrate) concurrently with 100-500 mcg of the PDE4 inhibitor roflumilast or an equivalent amount of a pharmaceutically acceptable salt of roflumilast is one such combination. This combination can both lead to greater improvement in depressive, anhedonia, motivational or cognitive symptoms and/or lead to lower side effects compared to use of a PDE4 inhibitor such as roflumilast alone.

β₃ adrenergic agonist and PDE4 inhibitor combination: The β₃ adrenergic receptor is another G-protein coupled receptor present in the brain, whose activation results in increased cAMP signaling. Most β₃ agonists are not brain-penetrant, and thus have been used for peripheral adrenergic stimulation, such as is the case for mirabegron which is approved for overactive bladder. Though another β₃ agonist, amibegron, is brain penetrant and had promising antidepressant-like effects in animals (25), it failed to show efficacy in two acute treatment clinical trials (as required by the U.S. Food and Drug Administration for demonstration of efficacy) and its development for the treatment of depression in humans was discontinued (see https://www.sanofi.com/en/science-and-innovation/clinical-trials-and-results/our-disclosure-commitments/pharma/letter-a (accessed on Jan. 12, 2021). Moreover, combination drugs including an β₃ agonist have not been tested for the treatment of depression. Thus, combination of a PDE4 inhibitor with a failed prospective antidepressant in order to become an effective treatment is unexpected. One embodiment of the invention is a combination of an β₃ agonist with a sub-emetic amount of a PDE4 inhibitor for treatment of symptoms related to depression, anhedonia, motivation-related or cognitive impairments. An example of an β₃ agonist is amibegron. Thus, for example, 100-1400 mg of amibegron, or an equivalent amount of a pharmaceutically acceptable salt thereof (e.g., amibegron hydrochloride), concurrently with 100-500 mcg of the PDE4 inhibitor roflumilast or an equivalent amount of a pharmaceutically acceptable salt of roflumilast is one such combination. This combination can both lead to greater improvement in depressive, anhedonia, motivational or cognitive symptoms and/or lead to lower side effects compared to use of a PDE4 inhibitor such as roflumilast alone.

TAAR1 agonist and PDE4 inhibitor combination: The trace amine-associated receptor 1 (TAAR1) is another G-protein coupled receptor present in the brain, whose activation results in increased cAMP signaling. Unlike the receptors above, it is located intracellularly and not on the cell surface. To date, no drug has been approved for any indication that specifically targets TAAR1 and activates it. One such TAAR1 agonist has shown initial promise in schizophrenia (26), but no TAAR1 agonist has been studied for efficacy in treating depression. Thus, a combination of a PDE4 inhibitor with a TAAR1 agonist is an unexpected combination for yielding an effective antidepressant. One embodiment of the invention is a combination of a TAAR1 agonist with a sub-emetic amount of a PDE4 inhibitor for the treatment of symptoms related to depression, anhedonia, motivation-related or cognitive impairments. An example of a TAAR1 agonist is ulotaront (SEP-363856). Another example is ralmitaront (RO6889450). Thus, for example, 5-200 mg of ulotaront (SEP-363856), or an equivalent amount of a pharmaceutically equivalent salt thereof, concurrently with 100-500 mcg of the PDE4 inhibitor roflumilast or an equivalent amount of a pharmaceutically acceptable salt of roflumilast is one such combination. In another example, 5-300 mg of ralmitaront (RO6889450), or an equivalent amount of a pharmaceutically equivalent salt thereof, concurrently with 100-500 mcg of the PDE4 inhibitor roflumilast or an equivalent amount of a pharmaceutically acceptable salt of roflumilast is one such combination. These combinations can both lead to greater improvement in depressive, anhedonia, motivational or cognitive symptoms and/or lead to lower side effects compared to use of a PDE4 inhibitor such as roflumilast alone.

Gene expression-based modeling of combination drug effects: To understand the potential utility of drug effects, the combinations described herein were assessed for two properties: complementary exposure and combination gene expression perturbations. While these properties and the results measuring these properties are described in the rest of the text, overall, these methods integrate mechanistic information about the drugs (e.g. the impact of the drug on gene expression levels in neural cells) with biological information about the indications (e.g. differences in gene expression levels between individuals with a given disease and without the disease). Each combination-indication pair was assessed for these two properties separately and shown here the results in tandem to support the utility of these drug effects.

Proximity with phenotype networks and separation between single drugs: Cellular systems operate as networks in which genes, their products, and other molecules interact to ensure proper cell function. Targeting a gene or protein has effects that extend throughout the molecular network to its downstream targets and can potentially affect entire pathways (29-30). Therefore, a systematic approach was used to analyze entire gene networks perturbed by the drug combinations. In the context of a biological network, genes within pathways perturbed by effective drugs are more likely to be closer to genes implicated in the disease and disease phenotypes than non-indicated drugs (30). Furthermore, effective drug combinations should follow a complementary exposure pattern, where each single drug targets genes that impact the disease module (where a module consists of a subnetwork where the nodes are genes implicated in disease), but different drugs target separate disease topological neighborhoods or sets of genes (31). For each combination, the proximity between the single-drug modules (where the modules consist of drug targets) and depression, cognition, PTSD, schizophrenia, addiction, Parkinson's disease, and bipolar disorder modules, as well as their separation from each other was evaluated. For this purpose, a brain-specific protein-protein interaction (PPi) network was constructed, where nodes represent genes expressed in the human brain and connections between nodes (referred to as “edges”) represent experimentally determined physical interactions between the gene products (31-33).

High-confidence disease-associated genes were identified based on mechanistic evidence from at least two published studies. All of the indications contain genes that are differentially expressed in post-mortem human brains. Additionally, for all indications except depression, genes from studies that were well supported with at least two lines of evidence were included. While one line of evidence could be genotype based (e.g. gene-based results from genome wide association studies), at least one line of evidence was also required to be functional evidence (e.g. expression quantitative trait loci, chromatin interaction studies, or gene expression studies in animal models). Finally, these gene lists were filtered to include only genes in the PPi network. The final sizes of the gene lists are as follows: 241 genes for depression (34-41), 470 genes for cognition (42-44), 26 genes for PTSD (45-55), 365 genes for schizophrenia (40, 56-64), 75 genes for addiction (55, 65-69), and 41 genes for Parkinson's disease (70-81).

To identify drug gene targets, direct target information was extracted from DGIdb (drug-gene interaction database (82)), and used these direct target nodes as “baits” by extending the module to include their first neighbors (defined as genes that are directly connected to the node in the network). The resulting modules were filtered to include only genes whose expression is impacted by the drugs of interest. These genes were assessed by gene expression data (83). Specifically, neural cell lines (neurons and neural precursor) were treated with different drugs and gene expression levels were measured post-treatment. Genes whose expression levels respond to treatment are hypothesized to be impacted (directly or indirectly) by the drug. Drugs for which gene expression data is available were chosen, with the same drug mechanism of action (MOA) as in the combinations. Specifically, gene expression data in response to rolipram was used to understand the effect of PDE4 inhibitors such as roflumilast, cisapride to understand the effect of 5-HT₄ agonists such as prucalopride (both 5-HT₄ agonists), ciproxifan to understand the effect of H₃ inverse agonists such as pitolisant or irdabisant, amibegron to understand the effect of (33 adrenergic agonists and tyramine to understand the effect of TAAR1 agonists such as SEP-363856 (ulotaront) and ralmitaront (RO6889450). For the β₃ adrenergic agonists, there is no gene expression data available in the Subramanian et al dataset, so only the direct targets of amibegron were used in the analyses. By analyzing drugs that are good representatives of the mechanisms of action of the drugs of interest, the results provide evidence for the utility of combinations of other drugs in those same classes when used in combination.

To assess proximity between genes targeted by the drugs of interest and genes whose expression is perturbed in disease, a permutation-based approach as described in Guney et al. 2016 (84) and Cheng et al. 2019 (31) for non-psychiatric indications was employed. Random modules matching the drug module for size and degree (as measured by the node's amount of connectivity, specifically the number of edges associated with it) are selected 1000 times and their distance to the disease module is computed to generate a distribution of distances. The proximity of the drug module to the disease module is given by a Z-score of its distance relative to the generated distribution based on random modules, approximated by (d_(drug-disease)-<d_(random-disease)>)/(σ(d_(random-disease))) where <d_(random-disease)> is the average of the distance distribution and σ(d_(random-disease)) the standard deviation. A Z-score<0 indicates the drug module is significantly more proximal to the disease module than other gene sets in the network. Indeed, in these analyses, Z-scores remain stable and consistently negative or positive across multiple permutation shuffles, indicating statistical significance of the findings regarding the combinations. The proximities were computed with all the disease and cognition modules for each drug.

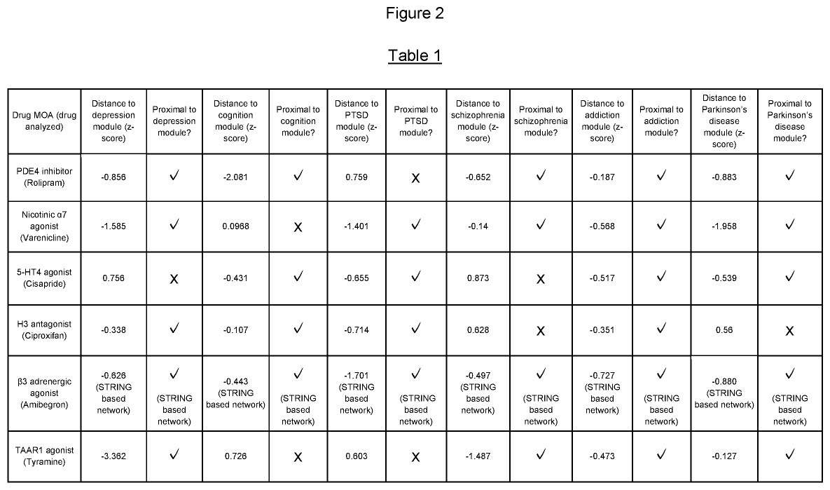

As shown in Table 1 (FIG. 2), all the drugs within the mechanistic combinations are proximal to multiple disease modules, supporting their indication in treating the corresponding conditions. The exception is amibegron, for which no gene expression data was available. This data unavailability initially resulted in a smaller, less complete drug network. To increase the robustness of the brain-specific PPi network, nodes and edges extracted from the STRING database were added (32). As demonstrated in Table 1, amibegron's direct targets module was also found to be proximal to all of the disease and trait modules tested. By comparing the results of drugs in the combinations to other gene sets in the network with similar properties, the results were demonstrated to be highly specific to the selected drugs.

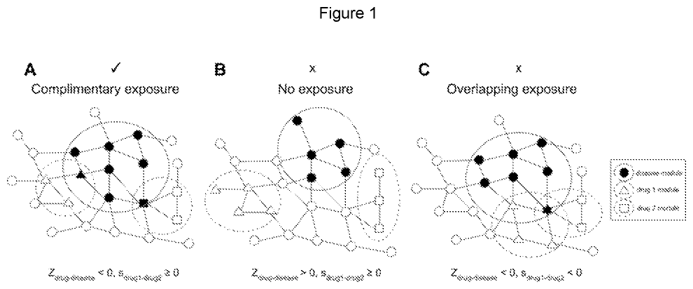

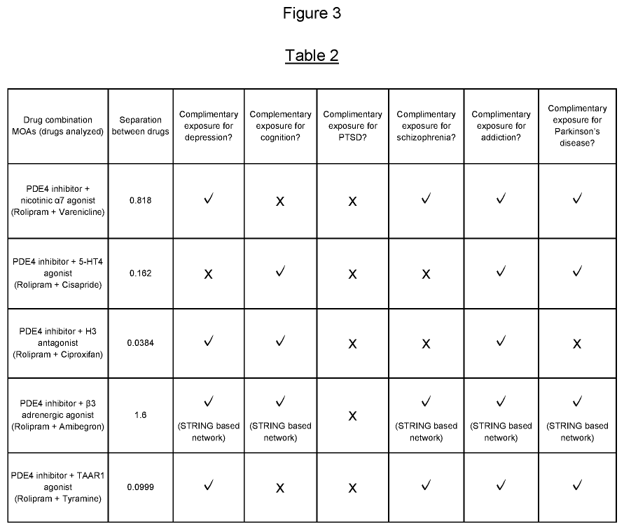

To evaluate separation between single drugs, the separation metric as described in Cheng et al. 2019 (31) was employed, which compares the distances between nodes within each module to the distances between modules. A zero or positive separation value indicates the two networks are topologically separated and thus hit different neighborhoods of the disease network, as shown in FIG. 1. Table 2 (FIG. 3) shows that within each proposed combination, single drugs are positively separated, that is, each drug within the combination is likely to target the depression gene set but different genes within the set.

These results show that the drug combinations follow the complimentary exposure pattern characteristic of combinations with high effectiveness and few adverse effects (31).

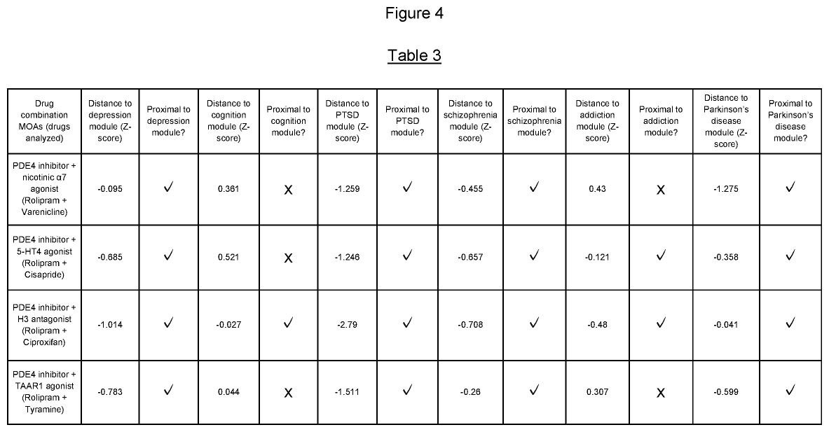

Prediction of perturbed networks based on gene expression: In addition to the complimentary exposure analysis, publicly-available gene expression data (83) was used to predict perturbation networks for each single drug and combination. Currently, there is no large-scale, readily-available gene expression data for combinations of drugs. Therefore, a nonlinear approach was used that integrates single-drug data to predict combination gene expression changes as described in Wu et al. 2010 (85), which was developed to identify combinations for type 2 diabetes. First, gene expression changes in neural cells after exposure to a single drug were quantified. Specifically, using the Subramanian et al. (83) dataset for neural cell drug dosing, the ratio of expression for each gene between drug treatment to no drug DMSO control for each single drug was computed. This information was used to predict how gene expression levels would change if exposed to two drugs at the same time, on a per-gene basis, in a metric called combination expression ratio. To compute this combination expression ratio, the computed single drug expression ratios for each gene were used. If the gene was downregulated upon treatment with each single drug, then the combination expression ratio was defined as the minimum value of the single drugs ratios. If the gene was upregulated in response to treatment of both drugs separately, the combination expression ratio was defined as the maximum value of the single drug ratios. If the ratios from each of the two drugs were in opposite directions, the combination expression ratio was computed as a sum of the single drug expression ratios (derived in Wu et al. 2010 (85)). A weight for each gene was then calculated by taking the absolute value of the logarithm of the computed expression ratios. A gene was considered as “perturbed” if its weight is greater or equal to 0.2, which is equivalent to a 20% single-drug-mediated up or downregulation of expression. For each single drug and combination, the perturbed genes were mapped to the brain-specific PPi network previously constructed and the proximity of the combination modules to the depression, cognition, PTSD, schizophrenia, addiction, and Parkinson's disease modules was evaluated. As shown in Table 3 (FIG. 4), the predicted combination networks are significantly proximal to multiple disease modules, supporting their indication in treating the corresponding conditions.

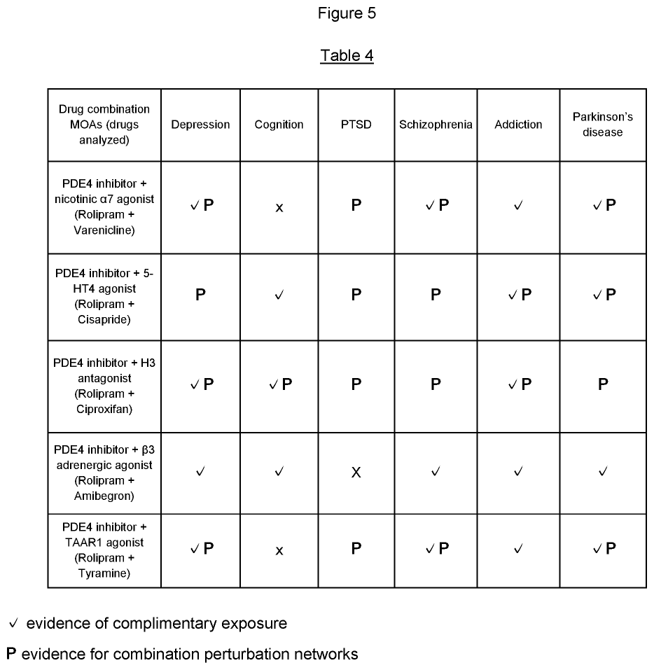

Summary of gene expression modeling: In Table 4 (FIG. 5) the findings from the two gene expression modeling methods above with respect to each of the combinations is summarized. Taken together, these results demonstrate that the combinations show significant evidence of complementary exposure and combination perturbation networks proximal to most, if not all, conditions in which depressive, anhedonia, motivation-related or cognition-related dysfunction exists.

### Definitions

“PDE4 inhibitor” refers to a compound that blocks or inhibits the activity of the phosphodiesterase 4 protein or any of its isoforms. Suitable PDE4 inhibitors antagonists include, but are not limited to, roflumilast, AVE8112, MEM1414, MEM1917, apremilast, cilomilast, crisaborole, ibudilast, luteolin, mesembrenon, piclamilast, rolipram, chlorbipram, GSK-256066, E-6005, MK-0873, BPN14770, HT-0712 and pharmaceutically acceptable salts thereof.

“5-HT₄ agonist” refers to an agonist of the 5-HT₄ receptor (including, but not limited to a 5-HT₄ partial agonist), and includes but is not limited to prucalopride, cisapride, BIMU-8, CJ-033466, ML-10302, mosapride, renzapride, RS-67506, RS67333, SL65.0155 (capeserod), tegaserod, zacopride, metoclopramide, supride and pharmaceutically acceptable salts thereof (such as prucalopride succinate or capeserod hydrochloride).

“H₃ antagonist” or “H₃ inverse agonist” refers to a compound that blocks activity at the H₃ receptor. Suitable H₃ antagonists or inverse agonists include, but are not limited to, pitolisant, ABT-28, BF2.649, CEP-26401 (irdabisant), GSK-189254, GSK-239512, MK-0249, PF-3654746 and pharmaceutically acceptable salts thereof (such as pitolisant hydrochloride or irdabisant hydrochloride).

“Nicotinic α₇ receptor agonist” or “alpha7 receptor agonist” refers to an agonist (including, but not limited to, a partial agonist) of the nicotinic receptor containing an α₇ subunit. Suitable α₇ nicotinic receptor agonist include, but are not limited to, varenicline, tilorone, A-582941, AR-R17779, TC-1698, bradanicline, encenicline, GTS-21, PHA-543613, PNU-292987, PHA-709829, SSR-180711, tropisetron, WAY-317538, anabasine, epiboxidine, PNU-120596, NS-1738, AVL-3288, A867744, ivermectine, BNC210 or a pharmaceutically acceptable salt thereof (such as varenicline tartrate).

“β₃ adrenergic agonist” or “β₃ agonist” refers to an agonist of the β₃ adrenergic receptor. Suitable β₃ agonists include amibegron, mirabegron, vibegron, ritobegron, BRL37344, solabegron, or a pharmaceutically acceptable salt thereof.

“TAAR1 agonist” refers to an agonist of the trace-amine associated receptor 1. Suitable TAAR1 agonists include ulotaront (SEP-363856), ralmitaront (RO6889450), RO5166017, RO5256390, RO5203648, RO5263397, tyramine, amphetamine, methamphetamine, and 3,4-methylenedioxy-methamphetamine (MDMA) or a pharmaceutically acceptable salt thereof.

Unless otherwise specified, the term “about” in the context of a numerical value or range refers to ±10% of the numerical value or range recited.

As used herein, “effective” as in an amount effective to achieve an end means the quantity of a component that is sufficient to yield an indicated therapeutic response without undue adverse side effects (such as toxicity, irritation, or allergic response) commensurate with a reasonable benefit/risk ratio when used in the manner of this disclosure. The specific effective amount varies with such factors as the particular condition being treated, the physical condition of the patient, the type of mammal being treated, the duration of the treatment, the nature of concurrent therapy (if any), and the specific formulations employed and the structure of the compounds or its derivatives.

As used herein, to “treat” or “treating” encompasses, e.g., inducing inhibition, regression, or stasis of a disorder and/or disease, e.g. depression, or alleviating, lessening, suppressing, inhibiting, reducing the severity of, eliminating or substantially eliminating, or ameliorating a symptom of the disease or disorder.

As used herein, the terms “subject” and “patient” are used interchangeably and refer to a human patient unless indicated otherwise.

Diagnosis of various mental and psychological disorders, including depression may be found, e.g., in the Diagnostic and Statistical Manual of Mental Disorders (5^{th} Ed. DSM-5, American Psychiatric Association, 2013).

Methods of Treatment

Each active ingredient (such as a PDE4 inhibitor, 5-HT₄ agonist, H₃ antagonist or inverse agonist, a nicotinic α₇ receptor agonist, a β₃ adrenergic agonist or a TAAR1 agonist) may be administered by any route, such as orally, nasally, transdermally, rectally, percutaneously or by parenteral injection. A preferred route of administration is oral. The active ingredients may be administered in the form of a tablet, capsule, granules, or oral liquid.

The methods and pharmaceutical compositions described herein may be used to treat (a) depression (such as major depressive disorder or bipolar I disorder), (b) a psychiatric or neurological disorder in which depressive, anhedonia, motivation-related or cognition-related dysfunction exists, or (c) one or more symptoms associated with depression, anhedonia, or motivation-related impairments. The types of depression which may be treated include, but are not limited to, major depressive disorder, treatment resistant depression, residual depressive symptoms and dysthymia. Psychiatric or neurological disorders in which depressive, anhedonia, motivation-related or cognitive-related dysfunction exists which may be treated include, but are not limited to, depression as part of bipolar I or bipolar II disorders, addiction (e.g., drug addiction), post-traumatic stress disorder, schizophrenia (in particular associated negative or cognitive symptoms) or Parkinson's Disease (non-motor features such as depression, apathy or cognitive impairment). In one embodiment, the method treats one or more non-motor features of Parkinson's disease. Symptoms associated with depression which may be treated include, but are not limited to, depressed mood, blunted affect, anhedonia, alexithymia, and apathy. Motivation-related impairments which may be treated include, but are not limited to, inability to engage in previously rewarding experiences, reduced social interest or drive, inattentiveness to social inputs, reduced psychomotor activity, excessive sleep, avoidance of activities or social interactions, and decreased appetite. Cognitive impairment includes, but is not limited to, inability to focus on attentionally-demanding tasks, poor executive functioning, difficulties in inhibiting inappropriate response and deficits in memory formation or recall.

Generally, the amount of the active ingredients to be administered is sufficient to increase cAMP molecular signaling in the brain. In one embodiment, the amount of each component to be administered daily can be as shown in the table below.

| Active Ingredient | Category | Range | Preferred Range |
|---|---|---|---|
| Roflumilast or a | PDE4 inhibitor | Amount equivalent to | Amount equivalent to |
| pharmaceutically | | about 100 mcg to 500 | about 100 mcg to 250 |
| acceptable salt thereof | | mcg roflumilast free | mcg roflumilast free |
| | | base daily (preferably | base daily (preferably |
| | | once daily) | once daily) |
| Varenicline or a | nicotinic α7 agonist | Amount equivalent to | Amount equivalent to |
| pharmaceutically | | about 0.25 to about 3 | 0.5 to 1 mg |
| acceptable salt thereof | | mg varenicline free | varenicline free base |
| (e.g., varenicline | | base daily (preferably | (e.g., 0.85 mg to 1.71 |
| tartrate) | | given once daily or in | mg of varenicline |
| | | two divided doses) | tartrate) daily |
| | | | (preferably given once |
| | | | daily or in two |
| | | | divided doses) |
| Prucalopride or a | 5-HT₄ agonist | Amount equivalent to | Amount equivalent to |
| pharmaceutically | | about 0.25 mg to | 0.5 to 1 mg |
| acceptable salt thereof | | about 4 mg | prucalopride free base |
| (e.g., prucalopride | | prucalopride free base | (e.g., 0.66 to 1.32 mg |
| succinate) | | daily (preferably | pricalopride |
| | | given once daily or in | succinate) daily |
| | | two divided doses) | (preferably given once |
| | | | daily or in two |
| | | | divided doses) |
| Capeserod or a | 5-HT₄ agonist | Amount equivalent to | Amount equivalent to |
| pharmaceutically | | about 1 μg to about 10 | 1 μg to 1 mg |
| acceptable salt thereof | | mg capeserod free | capeserod free base |
| (e.g., capeserod | | base daily (preferably | daily (preferably |
| hydrochloride) | | given once daily or in | given once daily or in |
| | | two divided doses) | two divided doses) |
| Pitolisant or a | H₃ antagonist or | An amount of | Amount equivalent to |
| pharmaceutically | inverse agonist | pitolisant or a | 4.45 mg to 17.8 mg |
| acceptable salt thereof | | pharmaceutically | pitolisant free base |
| (e.g., pitolisant | | acceptable salt thereof | (e.g., 5 to 20 mg of |
| hydrochloride) | | equivalent to about 2 | pitolisant |
| | | to about 40 mg of | hydrochloride) daily |
| | | pitolisant | (given once daily or in |
| | | hydrochloride daily | two divided doses) |
| | | (given once daily or in | |
| | | two divided doses) | |
| Irdabisant or a | H₃ antagonist or | An amount of | Amount equivalent to |
| pharmaceutically | inverse agonist | irdabisant or a | 5 μg to 250 μg |
| acceptable salt thereof | | pharmaceutically | irdabisant HCl daily |
| (e.g., irdabisant | | acceptable salt thereof | (given once daily or in |
| hydrochloride) | | equivalent to about | two divided doses) |
| | | 1 μg to about 500 μg | |
| | | of irdabisant | |
| | | hydrochloride daily | |
| | | (given once daily or in | |
| | | two divided doses) | |
| Amibegron or a | β₃ adrenergic agonist | An amount of | An amount of |
| pharmaceutically | | amibegron or a | amibegron or a |
| acceptable salt thereof | | pharmaceutically | pharmaceutically |
| (e.g., amibegron | | acceptable salt thereof | acceptable salt thereof |
| hydrochloride) | | (e.g., amibegron | (e.g., amibegron |
| | | hydrochloride) | hydrochloride) |
| | | equivalent to about | equivalent to 100 mg |
| | | 100 mg to 1400 mg of | to 700 mg of |
| | | amibegron daily in a | amibegron daily in a |
| | | single or two divided | single or two divided |
| | | doses | doses |
| ulotaront (SEP- | TAAR1 agonist | An amount of SEP- | An amount of SEP- |
| 363856), ralmitaront | | 363856 or a | 363856 or a |
| (RO6889450) or | | pharmaceutically | pharmaceutically |
| pharmaceutically | | acceptable salt thereof | acceptable salt thereof |
| acceptable salts | | equivalent to about | equivalent to about |
| thereof | | 5 mg to 200 mg daily | 5 mg to 150 mg daily |
| | | in a single or two | in a single or two |
| | | divided doses, or an | divided doses, or an |
| | | amount of ralmitaront | amount of ralmitaront |
| | | (RO6889450) or a | (RO6889450) or a |
| | | pharmaceutically | pharmaceutically |
| | | acceptable salt thereof | acceptable salt thereof |
| | | equivalent to about | equivalent to about |
| | | 5 mg to 300 mg daily | 5 mg to 200 mg daily |
| | | in a single or two | in a single or two |
| | | divided doses | divided doses |

In one embodiment, a sub-emetic amount of the PDE4 inhibitor is administered. In a preferred embodiment, an amount of the PDE4 inhibitor is administered which generally does not produce nausea or vomiting in the subject. In another embodiment, an amount of the PDE4 inhibitor is administered which does not evoke vomiting in the subject.

In accordance with the practice of the invention, each active ingredient can be administered one or more times a day, daily, weekly, monthly or yearly.

Pharmaceutical Compositions

The pharmaceutical composition can include a sub-emetic amount of the PDE4 inhibitor. In a preferred embodiment, the composition includes an amount of the PDE4 inhibitor which when administered (e.g., orally administered) generally does not produce nausea or vomiting in the subject. In another embodiment, the composition includes an amount of the PDE4 inhibitor which when administered (e.g., orally administered) does not evoke vomiting in the subject. In yet another embodiment, the pharmaceutical composition includes an amount of the PDE4 inhibitor and the other active ingredient as recited in the table provided above for one day. In yet another embodiment, the pharmaceutical composition includes an amount of the PDE4 inhibitor and the other active ingredient which when administered twice a day is equivalent to the total daily amount recited in the table provided above.

The pharmaceutical composition can include one or more pharmaceutically acceptable excipients in addition to the active ingredients. The pharmaceutical composition may be suitable for any route of administration, such as nasal, rectal, intercisternal, buccal, intramuscular, intrasternal, intracutaneous, intrasynovial, intravenous, intraperitoneal, intraocular, periosteal, intra-articular injection, infusion, oral, topical, inhalation, parenteral, subcutaneous, implantable pump, continuous infusion, gene therapy, intranasal, intrathecal, intracerebroventricular, transdermal, or by spray, patch or injection.

The pharmaceutical composition may be formulated as a solid dosage form, such as capsules, pills, soft-gels, tablets, caplets, troches, wafer, sprinkle, or chewing for oral administration. The pharmaceutical composition may also be formulated as a liquid dosage form such as an elixir, suspension or syrup.

The pharmaceutical composition may also be presented in a dosage form for transdermal application (e.g., a patch or an ointment) or oral administration.

The pharmaceutical composition may be in a liquid dosage form or a suspension to be applied to nasal cavity or oral cavity using a dropper, a sprayer or a container. The pharmaceutical composition may be in a solid, salt or powder to be applied to nasal cavity or oral cavity using a sprayer, forced air or a container.

The pharmaceutical acceptable excipient may be selected from pharmaceutically acceptable carriers, binders, diluents, adjuvants, or vehicles, such as preserving agents, fillers, polymers, disintegrating agents, glidants, wetting agents, emulsifying agents, suspending agents, sweetening agents, flavoring agents, perfuming agents, lubricating agents (such as magnesium stearate), acidifying agents, coloring agent, dyes, preservatives and dispensing agents. Such pharmaceutically acceptable excipients are described in the Handbook of Pharmaceutical Excipients, 6^{th} Ed., Pharmaceutical Press and American Pharmaceutical Association (2009).

Pharmaceutically acceptable carriers are generally non-toxic to recipients at the dosages and concentrations employed and are compatible with other ingredients of the formulation. Examples of pharmaceutically acceptable carriers include water, saline, dextrose solution, ethanol, polyols, vegetable oils, fats, ethyl oleate, liposomes, waxes polymers, including gel forming and non-gel forming polymers, and suitable mixtures thereof. The carrier may contain minor amounts of additives such as substances that enhance isotonicity and chemical stability. Such materials are non-toxic to recipients at the dosages and concentrations employed, and include buffers such as phosphate, citrate, succinate, acetic acid, and other organic acids or their salts; antioxidants such as ascorbic acid; low molecular weight (less than about ten residues) polypeptides, e.g., polyarginine or tripeptides; proteins, such as serum albumin, gelatin, or immunoglobulin; hydrophilic polymers such as polyvinylpyrrolidone; amino acids, such as glycine, glutamic acid, aspartic acid, or arginine; monosaccharides, disaccharides, and other carbohydrates including cellulose or its derivatives, glucose, mannose, or dextrins; chelating agents such as EDTA; sugar alcohols such as mannitol or sorbitol; counterions such as sodium; and/or nonionic surfactants such as polysorbates, poloxamers, or PEG.

Examples of binders include, but are not limited to, microcrystalline cellulose and cellulose derivatives, gum tragacanth, glucose solution, acacia mucilage, gelatin solution, molasses, polyvinylpyrrolidone, povidone, crospovidone, sucrose and starch paste.

Examples of diluents include, but are not limited to, lactose, sucrose, starch, kaolin, salt, mannitol and dicalcium phosphate.

Examples of excipients include, but are not limited to, starch, surfactants, lipophilic vehicles, hydrophobic vehicles, pregelatinized starch, microcrystalline cellulose, lactose, milk sugar, sodium citrate, calcium carbonate, and dicalcium phosphate. Typical excipients for dosage forms such as a soft-gel include gelatin for the capsule and oils such as soy oil, rice bran oil, canola oil, olive oil, corn oil, and other similar oils; glycerol, polyethylene glycol liquids, and vitamin E TPGS as a surfactant.

Examples of disintegrating agents include, but are not limited to, complex silicates, croscarmellose sodium, sodium starch glycolate, alginic acid, corn starch, potato starch, bentonite, methylcellulose, agar and carboxymethylcellulose.

Examples of glidants include, but are not limited to, colloidal silicon dioxide, talc, corn starch.

Examples of wetting agents include, but are not limited to, propylene glycol monostearate, sorbitan monooleate, diethylene glycol monolaurate and polyoxyethylene laural ether.

Examples of lubricants include magnesium or calcium stearate, sodium lauryl sulphate, talc, starch, lycopodium and stearic acid as well as high molecular weight polyethylene glycols.

### REFERENCES

1. Nelson J C. The STAR*D study: a four-course meal that leaves us wanting more. Am J Psychiatry. 2006; 163:1864-1866.
2. Fujita M, Richards E M, Niciu M J, Ionescu D F, Zoghbi S S, Hong J, Telu S, Hines C S, Pike V W, Zarate C A, Innis R B. cAMP signaling in brain is decreased in unmedicated depressed patients and increased by treatment with a selective serotonin reuptake inhibitor. Mol Psychiatry. 2017; 22:754-759.
3. Itoh T, Abe K, Hong J, Inoue O, Pike V W, Innis R B, Fujita M. Effects of cAMP-dependent protein kinase activator and inhibitor on in vivo rolipram binding to phosphodiesterase 4 in conscious rats. Synapse. 2010; 64:172-176.
4. Dwivedi Y, Rizavi H S, Conley R R, Roberts R C, Tamminga C A, Pandey G N. Altered gene expression of brain-derived neurotrophic factor and receptor tyrosine kinase B in postmortem brain of suicide subjects. Arch Gen Psychiatry. 2003; 60:804-815.
5. Dowlatshahi D, MacQueen G M, Wang J F, Young L T. Increased temporal cortex CREB concentrations and antidepressant treatment in major depression. Lancet. 1998; 352:1754-1755.
6. Reiach J S, Li P P, Warsh J J, Kish S J, Young L T. Reduced adenylyl cyclase immunolabeling and activity in postmortem temporal cortex of depressed suicide victims. J Affect Disord. 1999; 56:141-151.
7. Bolger G B. The PDE4 cAMP-Specific Phosphodiesterases: Targets for Drugs with Antidepressant and Memory-Enhancing Action. Adv Neurobiol. 2017; 17:63-102.
8. Bertolino A, Crippa D, di Dio S, Fichte K, Musmeci G, Porro V, Rapisarda V, Sastre-y-Hernandez M, Schratzer M. Rolipram versus imipramine in inpatients with major, “minor” or atypical depressive disorder: a double-blind double-dummy study aimed at testing a novel therapeutic approach. Int Clin Psychopharmacol. 1988; 3:245-253.
9. Hebenstreit G F, Fellerer K, Fichte K, Fischer G, Geyer N, Meya U, Sastre-y-Hernandez M, Schony W, Schratzer M, Soukop W, et al. Rolipram in major depressive disorder: results of a double-blind comparative study with imipramine. Pharmacopsychiatry. 1989; 22:156-160.
10. Scott A I, Perini A F, Shering P A, Whalley L J. In-patient major depression: is rolipram as effective as amitriptyline? Eur J Clin Pharmacol. 1991; 40:127-129.
11. Blokland A, Van Duinen M A, Sambeth A, Heckman P R A, Tsai M, Lahu G, Uz T, Prickaerts J, Van Duinen M A, Sambeth A, Heckman P R A, Smit S, Tsai M, Lahu G, Uz T, Blokland A, Prickaerts J, Gilleen J, Farah Y, Davison C, Kerins S, Valdearenas L, Uz T, Lahu G, Tsai M, Ogrinc F, Reichenberg A, Williams S C, Mehta M A, Shergill S S. Acute treatment with the PDE4 inhibitor roflumilast improves verbal word memory in healthy old individuals: a double-blind placebo-controlled study Acute administration of roflumilast enhances immediate recall of verbal word memory in healthy young adults
   An experimental medicine study of the phosphodiesterase-4 inhibitor, roflumilast, on working memory-related brain activity and episodic memory in schizophrenia patients. Neurobiol Aging. 2019; 77:37-43.
12. Van Duinen M A, Sambeth A, Heckman P R A, Smit S, Tsai M, Lahu G, Uz T, Blokland A, Prickaerts J. Acute administration of roflumilast enhances immediate recall of verbal word memory in healthy young adults. Neuropharmacology. 2018; 131:31-38.
13. Gilleen J, Farah Y, Davison C, Kerins S, Valdearenas L, Uz T, Lahu G, Tsai M, Ogrinc F, Reichenberg A, Williams S C, Mehta M A, Shergill S S. An experimental medicine study of the phosphodiesterase-4 inhibitor, roflumilast, on working memory-related brain activity and episodic memory in schizophrenia patients. Psychopharmacology (Berl). 2018.
14. Carpenter D O, Briggs D B, Knox A P, Strominger N. Excitation of area postrema neurons by transmitters, peptides, and cyclic nucleotides. J Neurophysiol. 1988; 59:358-369.
15. O'Donnell J M, Zhang H T. Antidepressant effects of inhibitors of cAMP phosphodiesterase (PDE4). Trends Pharmacol Sci. 2004; 25:158-163.
16. Spina D. PDE4 inhibitors: current status. Br J Pharmacol. 2008; 155:308-315.
17. Lucas G, Rymar V V, Du J, Mnie-Filali O, Bisgaard C, Manta S, Lambas-Senas L, Wiborg O, Haddjeri N, Piñeyro G, Sadikot A F, Debonnel G. Serotonin(4) (5-HT(4)) receptor agonists are putative antidepressants with a rapid onset of action. Neuron. 2007; 55:712-725.
18. Priem E, Van Colen I, De Maeyer J H, Lefebvre R A. The facilitating effect of prucalopride on cholinergic neurotransmission in pig gastric circular muscle is regulated by phosphodiesterase 4. Neuropharmacology. 2012; 62:2126-2135.
19. Pauwelyn V, Ceelen W, Lefebvre R A. Synergy between 5-HT(4) receptor stimulation and phosphodiesterase 4 inhibition in facilitating acetylcholine release in human large intestinal circular muscle. Neurogastroenterol Motil. 2018; 30.
20. Murphy S E, Wright L C, Browning M, Cowen P J, Harmer C J. A role for 5-HT(4) receptors in human learning and memory. Psychol Med. 2019:1-9.
21. Schwartz J C. The histamine H₃ receptor: from discovery to clinical trials with pitolisant. Br J Pharmacol. 2011; 163:713-721.
22. Sadek B, Saad A, Sadeq A, Jalal F, Stark H. Histamine H₃ receptor as a potential target for cognitive symptoms in neuropsychiatric diseases. Behav Brain Res. 2016; 312:415-430.
23. Ghamari N, Zarei O, Arias-Montaño J A, Reiner D, Dastmalchi S, Stark H, Hamzeh-Mivehroud M. Histamine H(3) receptor antagonists/inverse agonists: Where do they go? Pharmacol Ther. 2019; 200:69-84.
24. Cheng Q, Yakel J L. Activation of α₇ nicotinic acetylcholine receptors increases intracellular cAMP levels via activation of AC1 in hippocampal neurons. Neuropharmacology. 2015; 95:405-414.
25. Stemmelin J, Cohen C, Terranova J P, Lopez-Grancha M, Pichat P, Bergis O, Decobert M, Santucci V, Francon D, Alonso R, Stahl S M, Keane P, Avenet P, Scatton B, le Fur G, Griebel G. Stimulation of the beta3-Adrenoceptor as a novel treatment strategy for anxiety and depressive disorders. Neuropsychopharmacology. 2008; 33:574-587.
26. Koblan K S, Kent J, Hopkins S C, Krystal J H, Cheng H, Goldman R, Loebel A. A Non-D2-Receptor-Binding Drug for the Treatment of Schizophrenia. N Engl J Med. 2020; 382:1497-1506.
27. Fatemi S H, et al., Phosphodiesterase-4A expression is reduced in cerebella of patients with bipolar disorder, Psychiatr Genet. 2008, 18(6):282-8.
28. Heckman P R A, et al., PDE and cognitive processing: beyond the memory domain, Neurobiol Learn Mem., 2015, 119:108-22.
29. Kim, S. S., Dai, C., Hormozdiari, F., et al. Genes with High Network Connectivity Are Enriched for Disease Heritability. Am Jou Hum Gen 104 (5), 896-913 (2019). https://doi.org/10.1016/j.ajhg.2019.03.020
30. Zipp F, Ivinson A J, Haines J L, et al. Network-based multiple sclerosis pathway analysis with GWAS data from 15 000 cases and 30 000 controls. Am J Hum Genet 92: 854-865 (2013). https://doi.org/10.1016/j.ajhg.2013.04.019
31. Cheng, F., Kovacs, I. A. & Barabasi, A L. Network-based prediction of drug combinations. Nat Commun 10, 1197 (2019). https://doi.org/10.1038/s41467-019-09186-x
32. Szklarczyk D, Gable A L, Lyon D, et al. STRING v11: protein-protein association networks with increased coverage, supporting functional discovery in genome-wide experimental datasets. Nucleic Acids Res 47 (D1): D607-D613 (2019). https://doi.org/10.1093/nar/gky1131
33. Lonsdale, J., Thomas, J., Salvatore, M. et al. The Genotype-Tissue Expression (GTEx) project. Nat Genet 45, 580-585 (2013). https://doi.org/10.1038/ng.2653
34. Labonté, B., Engmann, O., Purushothaman, I. et al. Sex-specific transcriptional signatures in human depression. Nat Med 23, 1102-1111 (2017). https://doi.org/10.1038/nm.4386.
35. Wingo, T. S., Liu, Y., Gerasimov, E. S. et al. Brain proteome-wide association study implicates novel proteins in depression pathogenesis. Nat Neurosci 24, 810-817 (2021). https://doi.org/10.1038/s41593-021-00832-6.
36. Girgenti, M. J., Wang, J., Ji, D. et al. Transcriptomic organization of the human brain in post-traumatic stress disorder. Nat Neurosci 24, 24-33 (2021). https://doi.org/10.1038/s41593-020-00748-7.
37. Togichi, M., Iwamoto, K., Bundo, M. et al. Gene expression profiling of major depression and suicide in the prefrontal cortex of postmortem brains. Neuroscience Research 80 (2), 184-191 (2007). https://doi.org/10.1016/j.neures.2007.10.010.
38. Iwamoto, K., Kakiuchi, C., Bundo, M. et al. Molecular characterization of bipolar disorder by comparing gene expression profiles of postmortem brains of major mental disorders. Mol Psychiatry 9, 406-416 (2004). https://doi.org/10.1038/sj.mp.4001437.
39. Yin, H., Pantazatos, S. P., Galfalvy, H., et al. A Pilot Integrative Genomics Study of GABA and Glutamate Neurotransmitter Systems in Suicide, Suicidal Behavior, and Major Depressive Disorder. Am J Med Genet Part B 171B, 414-426 (2016). https://doi.org/10.1002/ajmg.b.32423.
40. Ramaker, R. C., Bowling, K. M., Lasseigne, B. N. et al. Post-mortem molecular profiling of three psychiatric disorders. Genome Med 9, 72 (2017). https://doi.org/10.1186/s13073-017-0458-5.
41. Kohen, R., Dobra, A., Tracy, J. et al. Transcriptome profiling of human hippocampus dentate gyrus granule cells in mental illness. Transl Psychiatry 4, e366 (2014). https://doi.org/10.1038/tp.2014.9.
42. Savage, J. E., Jansen, P. R., Stringer, S. et al. Genome-wide association meta-analysis in 269,867 individuals identifies new genetic and functional links to intelligence. Nat Genet 50, 912-919 (2018). https://doi.org/10.1038/s41588-018-0152-6.
43. Lam, M., Trampush, J. W., Yu, J., et al. Large-Scale Cognitive GWAS Meta-Analysis Reveals Tissue-Specific Neural Expression and Potential Nootropic Drug Targets. Cell Rep 21 (9), 2597-2613 (2017). https://doi.org/10.1016/j.celrep.2017.11.028.
44. Lam, M., Chen, C Y., Ge, T. et al. Identifying nootropic drug targets via large-scale cognitive GWAS and transcriptomics. Neuropsychopharmacol. 46, 1788-1801 (2021). https://doi.org/10.1038/s41386-021-01023-4.
45. Bharadwaj R A, Jaffe A E, Chen Q et al. Genetic risk mechanisms of posttraumatic stress disorder in the human brain. J Neurosci Res 96 (1): 21-30 (2018). https://doi.org/10.1002/jnr.23957.
46. Girgenti, M. J., Wang, J., Ji, D. et al. Transcriptomic organization of the human brain in post-traumatic stress disorder. Nat Neurosci 24, 24-33 (2021). https://doi.om/10.1038/s41593-020-00748-7.
47. Holmes S E, Girgenti M J, Davis M T, et al. Altered metabotropic glutamate receptor 5 markers in PTSD: In vivo and postmortem evidence. Proc Natl Acad Sci USA 114 (31): 8390-8395 (2017). https://doi.org/10.1073/pnas.1701749114.
48. Huckins L M, Chatzinakos X, Breen M S, et al. Analysis of Genetically Regulated Gene Expression Identifies a Prefrontal PTSD Gene, SNRNP35, Specific to Military Cohorts. Cell Rep 31 (9): 107716. https://doi.org/10.1016/j.celrep.2020.107716.
49. Morrison F G, Miller M W, Wolf E J, et al. Reduced interleukin 1A gene expression in the dorsolateral prefrontal cortex of individuals with PTSD and depression. Neurosci Lett 23: 204-209 (2019). https://doi.org/10.1016/ineulet.2018.10.027.
50. Nievergelt C M, Maihofer A X, Klengel T, et al. International meta-analysis of PTSD genome-wide association studies identifies sex- and ancestry-specific genetic risk loci. Nat Commun 10 (1): 4558 (2019). https://doi.org/10.1038/s41467-019-12576-w.
51. Logue M W, Zhou Z, Morrison F G, et al. Gene expression in the dorsolateral and ventromedial prefrontal cortices implicates immune-related gene networks in PTSD. Neurobiol of Stress 15: 100398 (2021). https://doi.org/10.1016/iynstr.2021.100398.
52. Stein M B, Levey D F, Cheng Z et al. Genome-wide association analyses of post-traumatic stress disorder and its symptom subdomains in the Million Veteran Program. Nat Genet 53 (2): 174-178 (2021). https://doi.org/10.1038/s41588-020-00767-x.
53. Stone L A, Girgenti M J, Wang J, et al. Cortical Transcriptomic Alterations in Association With Appetitive Neuropeptides and Body Mass Index in Posttraumatic Stress Disorder. Int J Neuropsychopharmacol 24:1461-1457 (2020). https://doi.org/10.1093/ijnp/pyaa072.
54. Su Y A, Wu J, Zhang L, et al. Dysregulated mitochondrial genes and networks with drug targets in postmortem brain of patients with posttraumatic stress disorder (PTSD) revealed by human mitochondria-focused cDNA microarrays. Int J Biol Sci 4 (4): 223-35 (2008). https://doi.org/10.7150/ijbs.4.223.
55. Wolf E J, Zhao X, Hawn S E, et al. Gene expression correlates of advanced epigenetic age and psychopathology in postmortem cortical tissue. Neurobiol Stress 15: 100371 (2021). https://doi.org/10.1016/1.ynstr.2021.100371.
56. Bowen E F W, Burgess J L, Granger R, et al. DLPFC transcriptome defines two molecular subtypes of schizophrenia. Transl Psychiatry 9 (1): 147 (2019). https://doi.org/10.1038/s41398-019-0472-z.
57. Carlstrom E L, Niazi A, Etemadikhah M, et al. Transcriptome Analysis of Post-Mortem Brain Tissue Reveals Up-Regulation of the Complement Cascade in a Subgroup of Schizophrenia Patients. Genes (Basel) 12 (8): 1242 (2021). https://doi.org/10.3390/genes12081242
58. Dobbyn A, Huckins L M, Boocock J, et al. Landscape of Conditional eQTL in Dorsolateral Prefrontal Cortex and Co-localization with Schizophrenia GWAS. Am J Hum Genet 102 (6): 1169-1184 (2018). https://doi.org/10.1016/j.ajhg.2018.04.011
59. Gandal M J, Zhang P, Hadjimichael E, et al. Transcriptome-wide isoform-level dysregulation in ASD, schizophrenia, and bipolar disorder. Science 362 (6420): eaat8127. https://doi.org/10.1126/science.aat8127
60. Manchia M, Piras I S, Huentelman M J, et al. Pattern of gene expression in different stages of schizophrenia: Down-regulation of NPTX2 gene revealed by a meta-analysis of microarray datasets. Eur Neuropsychopharmacol 27 (10): 1054-1063 (2017). https://doi.org/10.1016/j.euroneuro.2017.07.002
61. Maycox M, Kelly F, Taylor A, et al. Analysis of gene expression in two large schizophrenia cohorts identifies multiple changes associated with nerve terminal function. Mol Psychiatry 14 (12): 1083-94 (2009). https://doi.org/10.1038/mp.2009.18
62. Mistry M, Gillis J, Pavlidis P. Genome-wide expression profiling of schizophrenia using a large combined cohort. Mol Psychiatry 18 (2): 215-25 (2013). https://doi.org/10.1038/mp.2011.172
63. Santarelli D, Carroll A P, Cairns H M et al. Schizophrenia-associated MicroRNA-Gene Interactions in the Dorsolateral Prefrontal Cortex. Genomics Proteomics Bioinformatics 17 (6): 623-634 (2019). https://doi.org/10.1016/j.gpb.2019.10.003
64. Zhao Z, Xu J, Chen J, et al. Transcriptome sequencing and genome-wide association analyses reveal lysosomal function and actin cytoskeleton remodeling in schizophrenia and bipolar disorder. Mol Psychiatry 20 (5): 563-572 (2015). https://doi.org/10.1038/mp.2014.82 Albertson D N, Schmidt C J, Kapatos G et al. Distinctive profiles of gene expression in the human nucleus accumbens associated with cocaine and heroin abuse. Neuropsychopharmacology 31 (10): 2304-12 (2006). https://doi.org/0.1038/sj.npp.1301089
65. Cabrera-Mendoza B, Martinez-Magana J J, Monroy-Jaramillo N, et al. Candidate pharmacological treatments for substance use disorder and suicide identified by gene co-expression network-based drug repositioning. Am J Med Genet B Neuropsychiatr Genet 186 (3): 193-206 (2021). https://doi.org/10.1002/ajmg.b.32830
66. Liu J, Lewohl J M, Harris R A, et al. Patterns of gene expression in the frontal cortex discriminate alcoholic from nonalcoholic individuals. Neuropsychopharmacology 31 (7): 1574-82 (2006).
67. Marees A T, Gamazon E R, Gerring Z et al. Post-GWAS analysis of six substance use traits improves the identification and functional interpretation of genetic risk loci. Drug Alcohol Depend 206: 107703 (2020). https://doi.org/10.1016/j.drugalcdep.2019.107703
68. McClintick J N, Xuei X, Tischfield J A, et al. Stress-response pathways are altered in the hippocampus of chronic alcoholics. Alcohol 47 (7): 505-15 (2013). https://doi.org/10.1016/j.alcohol.2013.07.002
69. Thompson A, Cook J, Choquet H et al., Functional validity, role, and implications of heavy alcohol consumption genetic loci. Sci Adv 6 (3): eaay5034 (2020). https://doi.org/0.1126/sciadv.aay5034
70. Bandres-Ciga, Saez-Atienzar S, Kim J J, et al. Large-scale pathway specific polygenic risk and transcriptomic community network analysis identifies novel functional pathways in Parkinson disease. Acta Neuropathol 140 (3): 341-358 (2020). https://doi.org/10.1007/s00401-020-02181-3
71. Chi J, Xie Q, Jia J, et al. Integrated Analysis and Identification of Novel Biomarkers in Parkinson's Disease. Front Aging Neurosci 10: 178 (2018). https://doi.org/10.3389/fnagi.2018.00178
72. Day J O, Mullin S. The Genetics of Parkinson's Disease and Implications for Clinical Practice. Genes (Basel) 12 (7): 1006 (2021). https://doi.org/10.3390/genes12071006
73. de Paiva Lopes, Snijders G J L, Humphrey J et al. Genetic analysis of the human microglial transcriptome across brain regions, aging and disease pathologies. Nat Genet online ahead of print. https://doi.org/0.1038/s41588-021-009′76-y
74. Donega V, Burm S M, van Strien M E, et al. Transcriptome and proteome profiling of neural stem cells from the human subventricular zone in Parkinson's disease. Acta Neuropathol Commun 7 (1): 84 (2019). https://doi.org/10.1186/s40478-019-0736-0
75. Germer E L, Imhoff S, C, et al. The Role of Rare Coding Variants in Parkinson's Disease GWAS Loci. Front Neurol 10: 1284 (2019). https://doi.org/10.33.89/fneur.2019.01284
76. Kia D A, Zhang D, Guelfi S, et al. Identification of Candidate Parkinson Disease Genes by Integrating Genome-Wide Association Study, Expression, and Epigenetic Data Sets. JAMA Neurol 78 (4): 464-472 (2021). https://doi.org/10.1001/jamaneurol.2020.5257
77. Nalls M A, Blauwendraat C, Vallerga C L, et al. Identification of novel risk loci, causal insights, and heritable risk for Parkinson's disease: a meta-analysis of genome-wide association studies. Lancet Neurol 18 (12): 1091-1102. Https://doi.org/10.1016/S1474-4422(19)30320-5
78. Nido G S, Dick F, Toker L, et al. Common gene expression signatures in Parkinson's disease are driven by changes in cell composition. Acta Neuropathol Commun 8 (1): 55 (2020). https://doi.org/10.1186/s40478-020-00932-7
79. Smajić S, Prada-Medina C A, Landoulsi Z, et al. Single-cell sequencing of human midbrain reveals glial activation and a Parkinson-specific neuronal state. Brain online ahead of print. https://doi.org/10.1093/brain/awab446
80. Storm C S, Kia D A, Almramhi M M, et al. Finding genetically-supported drug targets for Parkinson's disease using Mendelian randomization of the druggable genome. Nat Commun 12 (1): 7342. https://doi.org/10.1038/s41467-021-26280-1
81. Tan M M X, Malek N, Lawton M A, et al. Genetic analysis of Mendelian mutations in a large UK population-based Parkinson's disease study. Brain 142 (9): 2828-2844 (2019). https://doi.org/10.1093/brain/awz191
82. Griffith M, Griffith O L, Coffman A C, et al. DGIdb: mining the druggable genome. Nat Methods 10 (12): 1209-10 (2013). https://doi.org/10.1038/nmeth.2689
83. Subramanian A, Narayan R, Corsello S M, et al. A Next Generation Connectivity Map: L1000 platform and the first 1,000,000 profiles. Cell 171 (6): 1437-1452.e17 (2017). https://doi.org/10.1016/j.cell.2017.10.049
84. Guney, E., Menche, J., Vidal, M. et al. Network-based in silico drug efficacy screening. Nat Commun 7, 10331 (2016). https://doi.org/10.1038/ncomms10331
85. Wu Z, Zhao X, Chen L. A systems biology approach to identify effective cocktail drugs. BMC Syst Biol 4 (Suppl 2): S7 (2010). https://doi.org/10.1186/1752-0509-4-S2-S7

All references cited herein are hereby incorporated by reference.

1. A pharmaceutical composition comprising a PDE4 inhibitor and at least one of a 5-HT₄ agonist, an H₃ antagonist or inverse agonist, or a nicotinic α₇ receptor agonist, a β₃ adrenergic agonist or a TAAR1 agonist.

2. The pharmaceutical composition of claim 1, wherein the composition comprises (a) a PDE4 inhibitor and (b) a 5-HT₄ agonist.

3. The pharmaceutical composition of claim 2, wherein the composition comprises (a) roflumilast, its N-oxide, or a pharmaceutically acceptable salt thereof and (b) prucalopride or a pharmaceutically acceptable salt thereof.

4. (canceled)

5. The pharmaceutical composition of claim 2, wherein the composition comprises (a) roflumilast, its N-oxide, or a pharmaceutically acceptable salt thereof and (b) capeserod or a pharmaceutically acceptable salt thereof.

6. (canceled)

7. The pharmaceutical composition of claim 1, wherein the composition comprises (a) a PDE4 inhibitor and (b) an H₃ antagonist or inverse agonist.

8. The pharmaceutical composition of claim 7, wherein the composition comprises (a) roflumilast, its N-oxide, or a pharmaceutically acceptable salt thereof and (b) pitolisant or a pharmaceutically acceptable salt thereof.

9. (canceled)

10. The pharmaceutical composition of claim 7, wherein the composition comprises (a) roflumilast, its N-oxide, or a pharmaceutically acceptable salt thereof and (b) irdabisant or a pharmaceutically acceptable salt thereof.

11. (canceled)

12. The pharmaceutical composition of claim 1, wherein the composition comprises (a) a PDE4 inhibitor and (b) a nicotinic α₇ receptor agonist.

13. The pharmaceutical composition of claim 12, wherein the composition comprises (a) roflumilast, its N-oxide, or a pharmaceutically acceptable salt thereof and (b) varenicline or a pharmaceutically acceptable salt thereof.

14. (canceled)

15. The pharmaceutical composition of claim 1, wherein the composition comprises (a) a PDE4 inhibitor and (b) a β₃ adrenergic agonist.

16. The pharmaceutical composition of claim 15, wherein the composition comprises (a) roflumilast, its N-oxide, or a pharmaceutically acceptable salt thereof and (b) amibegron or a pharmaceutically acceptable salt thereof.

17. (canceled)

18. The pharmaceutical composition of claim 1, wherein the composition comprises (a) a PDE4 inhibitor and (b) a TAAR-1 agonist.

19. The pharmaceutical composition of claim 18, wherein the composition comprises (a) roflumilast, its N-oxide, or a pharmaceutically acceptable salt thereof and (b) ulotaront or a pharmaceutically acceptable salt thereof.

20. (canceled)

21. The pharmaceutical composition of claim 18, wherein the composition comprises (a) roflumilast, its N-oxide, or a pharmaceutically acceptable salt thereof and (b) ralmitaront or a pharmaceutically acceptable salt thereof.

22. (canceled)

23. The pharmaceutical composition of claim 1, wherein the composition comprises a sub-emetic amount of component (a) and an effective amount of components (a) and (b) together to treat the intended disorder, such as (a) depression (such as major depressive disorder or bipolar I disorder), (b) a psychiatric or neurological disorder in which anhedonia, motivation-related or cognition-related dysfunction exists, or (c) one or more symptoms associated with depression, anhedonia, or motivation-related or cognition-related impairments.

24. A method of treating (a) depression, (b) a psychiatric or neurological disorder in which anhedonia, motivation-related or cognition-related dysfunction exists, or (c) one or more symptoms associated with depression, anhedonia, or motivation-related or cognition-related impairments in a subject in need thereof comprising administering to the subject an effective amount of a PDE4 inhibitor and at least one of a 5-HT₄ agonist, an H₃ antagonist or inverse agonist, a nicotinic α₇ receptor agonist, a β₃ adrenergic agonist or a TAAR1 agonist.

25. The method of claim 24, wherein the method comprises administering an effective amount of (a) a PDE4 inhibitor and (b) a 5-HT₄ agonist.

26. The method of claim 25, wherein the method comprises administering an effective amount of (a) roflumilast, its N-oxide, or a pharmaceutically acceptable salt thereof and (b) prucalopride or a pharmaceutically acceptable salt thereof.

27. (canceled)

28. The method of claim 25, wherein the method comprises administering an effective amount of (a) roflumilast, its N-oxide, or a pharmaceutically acceptable salt thereof and (b) capeserod or a pharmaceutically acceptable salt thereof.

29. (canceled)

30. The method of claim 24, wherein the method comprises administering an effective amount of (a) a PDE4 inhibitor and (b) an H₃ antagonist or inverse agonist.

31. The method of claim 30, wherein the method comprises administering an effective amount of (a) roflumilast, its N-oxide, or a pharmaceutically acceptable salt thereof and (b) pitolisant or a pharmaceutically acceptable salt thereof.

32. (canceled)

33. The method of claim 30, wherein the composition method comprises administering an effective amount of (a) roflumilast, its N-oxide, or a pharmaceutically acceptable salt thereof and (b) irdabisant or a pharmaceutically acceptable salt thereof.

34. (canceled)

35. The method of claim 24, wherein the method comprises administering an effective amount of (a) a PDE4 inhibitor and (b) a nicotinic α₇ receptor agonist.

36. The method of claim 35, wherein the method comprises administering an effective amount of (a) roflumilast, its N-oxide, or a pharmaceutically acceptable salt thereof and (b) varenicline or a pharmaceutically acceptable salt thereof.

37. (canceled)

38. The method of claim 24, wherein the method comprises administering an effective amount of (a) a PDE4 inhibitor and (b) a β₃ adrenergic agonist.

39. The method of claim 38, wherein the method comprises administering an effective amount of (a) roflumilast, its N-oxide, or a pharmaceutically acceptable salt thereof and (b) amibegron or a pharmaceutically acceptable salt thereof.

40. (canceled)

41. The method of claim 24, wherein the method comprises administering an effective amount of (a) a PDE4 inhibitor and (b) a TAAR-1 agonist.

42. The method of claim 41, wherein the method comprises administering an effective amount of (a) roflumilast, its N-oxide, or a pharmaceutically acceptable salt thereof and (b) ulotaront or a pharmaceutically acceptable salt thereof.

43. (canceled)

44. The method of claim 41, wherein the method comprises administering an effective amount of (a) roflumilast, its N-oxide, or a pharmaceutically acceptable salt thereof and (b) ralmitaront or a pharmaceutically acceptable salt thereof.

45. (canceled)

46. The method of claim 1, wherein the method comprises administering a sub-emetic amount of component (a) and an effective amount of components (a) and (b) together to treat the (a) depression (such as major depressive disorder or bipolar I disorder), (b) a psychiatric or neurological disorder in which anhedonia, motivation-related or cognition-related dysfunction exists, or (c) one or more symptoms associated with depression, anhedonia, or motivation-related or cognition-related impairments.

47. The method of claim 24, wherein the psychiatric or neurological disorder is post-traumatic stress disorder, schizophrenia, addiction, or Parkinson's disease.

48. The method of claim 24, wherein the psychiatric or neurological disorder is one or more non-motor features of Parkinson's disease.
